(19) United States
(12) Patent Application Publication
(10) Pub. No.: US 2026/0248966 A1
(43) Pub. Date: Aug. 27, 2026

(54) USE OF FCGAMMARIIB AS IGG TRANSPORT RECEPTOR IN MAMMARY GLAND OF MAMMALS

(71) Applicant: CHINA AGRICULTURAL UNIVERSITY, Beijing (CN)

(72) Inventors: Yaofeng ZHAO, Beijing (CN); Xin LI, Beijing (CN); Lijuan DU, Beijing (CN); Zihui WAN, Beijing (CN); Dengke PAN, Beijing (CN); Haitang HAN, Beijing (CN); Ran ZHANG, Beijing (CN)

(21) Appl. No.: 19/449,378

(22) Filed: Jan. 14, 2026

Related U.S. Application Data
(63) Continuation of application No. PCT/CN2025/081197, filed on Mar. 7, 2025.

(30) Foreign Application Priority Data
Feb. 24, 2025 (CN) 202510204364.X

(51) Int. Cl. A61K 48/00; A61K 38/17

(52) U.S. Cl. CPC A61K 48/005; A61K 38/1774

(57) ABSTRACT

The present application discloses use of FcgammaRIIB as an IgG transport receptor in mammary gland of mammals, and belongs to the field of biotechnology. FcgammaRIIB knockout mice and FcgammaRIIB knockout pigs are constructed, and the ratio of mammalian serum IgG to milk IgG in the FcgammaRIIB knockout mice is significantly increased, indicating a decrease in relative milk IgG concentration. Although mammalian serum IgG content is significantly increased in the FcgammaRIIB knockout pigs, IgG in colostrum of mammals is nearly undetectable. Therefore, the present application demonstrates for the first time that FcgammaRIIB serves as the receptor mediating transport of mammalian serum IgG across the mammary epithelial barrier into milk, which addresses a long-standing unresolved issue in the field of maternal passive immunity. Furthermore, the present application provides a new strategy for increasing colostrum IgG content, thereby enhancing early immunity and survival rate of newborn livestock.

### CROSS-REFERENCE TO RELATED APPLICATIONS

This application is a continuation of International Application No. PCT/CN2025/081197, filed on Mar. 7, 2025, which claims priority to Chinese Patent Application No. 202510204364.X, filed on Feb. 24, 2025, which is hereby incorporated by reference in its entirety.

### TECHNICAL FIELD

The present application relates to the field of biotechnology, and specifically to use of FcgammaRIIB as an IgG transport receptor in mammary gland of mammals.

### SEQUENCE LISTING

The present application contains a sequence listing which was filed electronically in XML format and is hereby incorporated by reference in its entirety. Besides, the XML copy is created on Jan. 13, 2026, is named “PA-2026010100-US-Sequence Listing” and is 21,119 bytes in sizes.

### BACKGROUND

Maternal IgG is crucial for optimal growth of offspring. Offspring acquire maternal IgG via two pathways: a first pathway involves transport through the placenta before birth, where maternal IgG directly enters fetal blood circulation; and a second pathway involves ingestion of milk after birth, where maternal IgG is transported from serum to milk and absorbed in the small intestine after milk ingestion by offspring. The process of crossing cellular layer barriers has been generally recognized as receptor-mediated. The placental cellular layer barrier and the small intestinal cellular layer barrier have been identified as being mediated by neonatal Fc receptor (FcRn). However, the receptor mediating IgG transport across mammary epithelial barriers remains unclear. In the prior art, FcRn has been identified as the transport receptor for passive transport of maternal IgG across placental and small intestinal cellular layer barriers. FcRn is not the receptor mediating maternal IgG transport across mammary epithelial barriers. Therefore, identification of the passive transport receptor for maternal IgG remains incomplete. Transport across the mammary gland represents the only pathway by which offspring of large animals acquire maternal IgG, and therefore identification of the receptor for this barrier transport is of significant importance.

### SUMMARY

To address the above defects in the prior art, the present application provides use of FcgammaRIIB as an IgG transport receptor in mammary gland of mammals to increase IgG content in mammalian milk.

A technical solution adopted by the present application for solving the above technical problem is as follows. Use of an FcgammaRIIB gene or a protein encoded thereby as an IgG transport receptor in mammary gland of mammals, where the FcgammaRIIB gene or the protein encoded thereby mediates transport of mammalian serum IgG across the mammary gland into milk.

Further, the mammals include mouse, rat, pig, cow, and sheep.

Further, mouse FcgammaRIIB has a nucleotide sequence set forth in SEQ ID NO. 1; rat FcgammaRIIB has a nucleotide sequence set forth in SEQ ID NO. 2; pig FcgammaRIIB has a nucleotide sequence set forth in SEQ ID NO. 3; cow FcgammaRIIB has a nucleotide sequence set forth in SEQ ID NO. 4; sheep FcgammaRIIB has a nucleotide sequence set forth in SEQ ID NO. 5;
a protein encoded by the mouse FcgammaRIIB has an amino acid sequence set forth in SEQ ID NO. 6; a protein encoded by the rat FcgammaRIIB has an amino acid sequence set forth in SEQ ID NO. 7; a protein encoded by the pig FcgammaRIIB has an amino acid sequence set forth in SEQ ID NO. 8; a protein encoded by the cow FcgammaRIIB has an amino acid sequence set forth in SEQ ID NO. 9; and a protein encoded by the sheep FcgammaRIIB has an amino acid sequence set forth in SEQ ID NO. 10.

Further, the IgG includes at least one of mouse IgG subtypes IgG1, IgG2a, IgG2b, IgG2c, and IgG3; IgG includes at least one of rat IgG subtypes IgG1, IgG2a, IgG2b, and IgG2c; IgG includes at least one of pig IgG subtypes IgG1, IgG2, IgG3, IgG4, and IgG5; IgG includes at least one of cow IgG subtypes IgG1 and IgG2; and IgG includes at least one of sheep IgG subtypes IgG1, IgG2, and IgG3.

The present application provides a method for increasing IgG content in mammalian milk, which includes overexpressing FcgammaRIIB in mammary tissue to increase IgG content in mammalian milk.

Further, overexpression of FcgammaRIIB includes the following steps: constructing an FcgammaRIIB mammary-specific overexpression vector, followed by microinjection of the overexpression vector.

Further, a backbone of the overexpression vector is a pBC1 expression vector.

The present application has the following beneficial effects: FcgammaRIIB knockout mice and FcgammaRIIB knockout pigs are constructed, and the ratio of mammalian serum IgG to milk IgG in the FcgammaRIIB knockout mice is significantly increased, indicating a decrease in relative milk IgG concentration. Although mammalian serum IgG content is significantly increased in the FcgammaRIIB knockout pigs, IgG in colostrum of mammals is nearly undetectable. Therefore, the present application demonstrates for the first time that FcgammaRIIB serves as the receptor mediating transport of mammalian serum IgG across the mammary epithelial barrier into milk, which addresses a long-standing unresolved issue in the field of maternal passive immunity. FcgammaRIIB is specifically overexpressed in mammary gland of mice to obtain FcgammaRIIB overexpressing mice, and IgG content in milk of the overexpressing mice is significantly increased while mammalian serum IgG remains nearly unchanged, indicating enrichment of IgG in milk of the overexpressing mice. The present application provides a new strategy for enhancing early immunity and survival rate of newborn livestock.

### BRIEF DESCRIPTION OF THE DRAWINGS

FIG. 1 shows the construction strategy of Fcgr1 and Fcgr4 knockout mice;

FIG. 2 shows detection of milk IgG transport in Fcgr2b knockout mice;

FIG. 3 shows detection of milk IgG transport in Fcgr2b conditional knockout mice;

FIG. 4 shows expression of FcgammaRIIB in mammary gland of pigs during the perinatal period;

FIG. 5 shows a map of PX458 vector;

FIG. 6 shows construction of FcgammaRIIB knockout pig model;

FIG. 7 shows detection of milk IgG transport in FcgammaRIIB knockout pigs;

FIG. 8 shows a map of pBC1-FcgammaRIIB1/FcgammaRIIB2 expression vector;

FIG. 9 shows detection of qPCR of FcgammaRIIB in mammary gland of transgenic mice; and

FIG. 10 shows detection of milk IgG transport in FcgammaRIIB overexpressing mice.

### DESCRIPTION OF EMBODIMENTS

The following examples are provided for illustrating the present application and are not intended to limit the scope of the present application. Where specific conditions are not indicated in the examples, conventional conditions or conditions recommended by the manufacturer are used. Reagents or instruments for which the manufacturer is not specified are conventional products commercially available.

### Example 1: Preparation of Fc γ Rs Knockout Mice and Detection of Milk IgG Transport

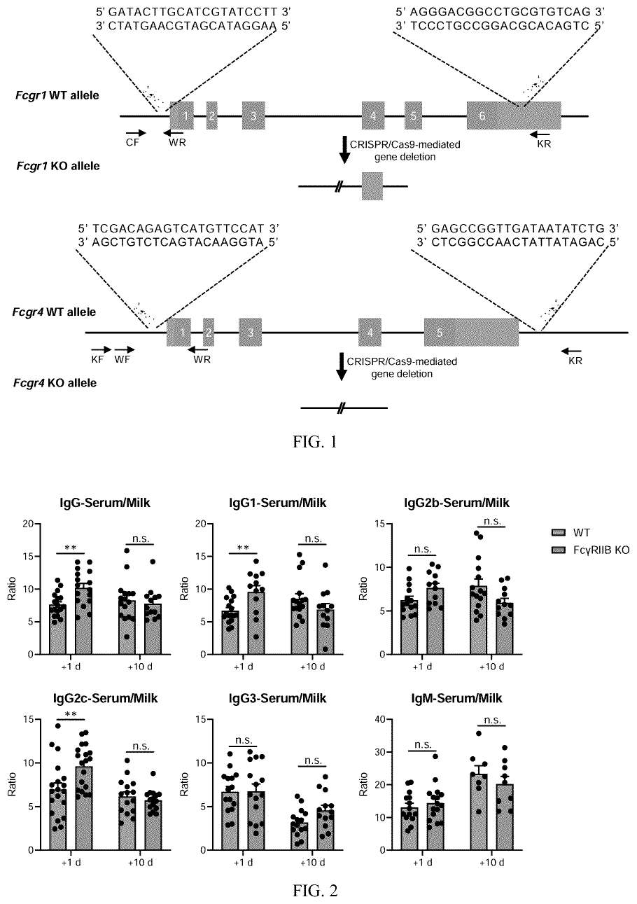

In mice, in addition to FcgammaRIIB, which belongs to the same receptor family, there are three other receptors, namely Fc ^{γ} I, Fc ^{γ} RIII, and Fc ^{γ} RIV. Therefore, mouse models with individual knockout of the four receptor genes (see FIG. 1) were used to identify the IgG transport receptor in mammary gland of mice. FcgammaRIIB knockout mice and Fc ^{γ} RIII knockout mice were purchased from Jackson Lab. Fc ^{γ} I knockout mice and Fc ^{γ} RIV knockout mice were constructed as follows: four gRNAs were designed for each of the mouse Fcgr1 and Fcgr4 gene sequences, with two gRNAs designed for each target site to be cleaved, prioritizing gRNAs with higher off-target scores. A DNA fragment of Fcgr4-sgRNA was amplified using sgRNA-pX330 as a template, followed by gel extraction to serve as a template for in vitro transcription of sgRNA, and then in vitro transcription and purification of sgRNA were performed. The purified sgRNA and Cas9 mRNA were co-injected into C57 mouse embryos. After injection, the embryos were transferred into the oviducts of surrogate recipient mice, and offspring were obtained. The nucleotide sequences of the four gRNAs are set forth as follows:

Fcgr1-gRNA1:
(SEQ UID NO. 11)
5′-GATACTTGCATCGTATCCTT-3′;
Fcgr1-gRNA2:
(SEQ UID NO. 12)
5′-CTGACACGCAGGCCGTCCCT-3′;
Fcgr4-gRNA1:
(SEQ UID NO. 13)
5′-ATGGAACATGACTCTGTCGA-3′;
Fcgr4-gRNA2:
(SEQ UID NO. 14)
5′-GAGCCGGTTGATAATATCTG-3′.

IgG content in postpartum serum and milk of the four knockout mice described above was measured by sandwich ELISA. The ratio of serum IgG to milk IgG (Ratio-Serum/Milk) was analyzed to evaluate milk IgG transport, and the same method was used in subsequent examples for measurement and analysis. The results showed that only FcgammaRIIB knockout mice exhibited a significantly increased ratio of serum IgG to milk IgG, indicating a decrease in relative milk IgG concentration (see FIG. 2). This suggests that FcgammaRIIB may mediate transport of mouse serum IgG into milk.

### Example 2: Preparation of FcgammaRIIB Conditional Knockout Mice and Detection of Milk IgG Transport

Since changes in serum IgG content in FcgammaRIIB knockout mice may affect milk IgG content, a mammary gland-specific FcgammaRIIB knockout mouse model was constructed using the Cre/LoxP system. MMTV-Cre and K14-Cre driver mice were crossed with FcgammaRIIB Flox mice to specifically delete Fcgr2b in mammary luminal epithelial cells and myoepithelial cells (MMTV-Cre mice were purchased from Cyagen Biosciences, and K14-Cre mice and FcgammaRIIB Flox mice were provided by other research groups).

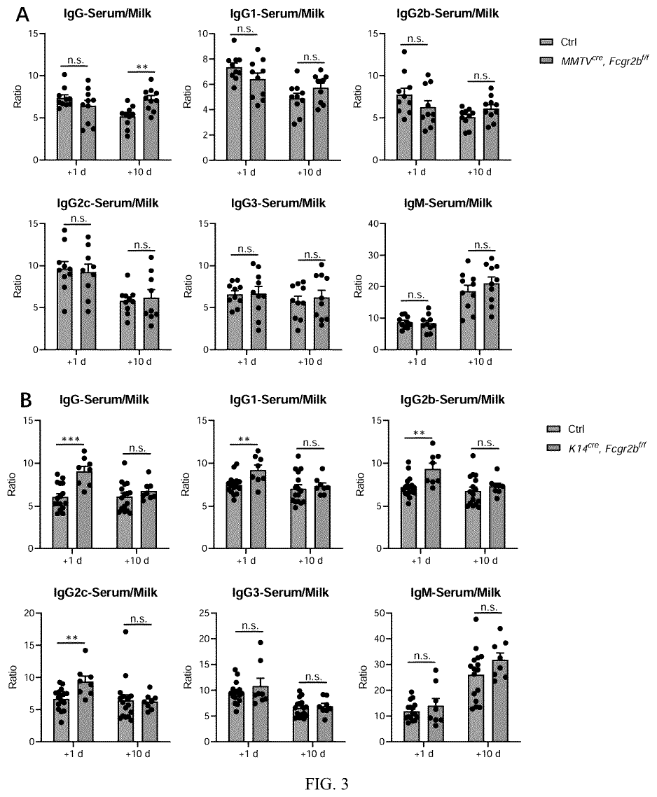

IgG content in postpartum serum and milk of the FcgammaRIIB conditional knockout mice was measured by sandwich ELISA. The ratio of serum IgG to milk IgG was analyzed to evaluate milk IgG transport. The results showed that serum IgG content in the mice did not change significantly, whereas milk IgG concentration was significantly reduced, and the ratio of serum IgG to milk IgG was significantly increased, indicating a decrease in relative milk IgG concentration (see FIG. 3). This suggests that FcgammaRIIB mediates transport of mouse serum IgG into milk.

### Example 3: Construction of FcgammaRIIB Knockout Pigs and Detection of Colostrum IgG Transport

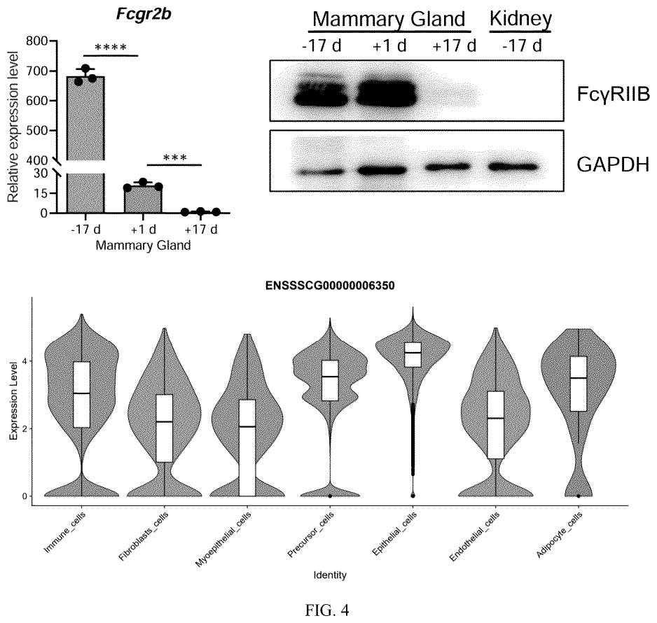

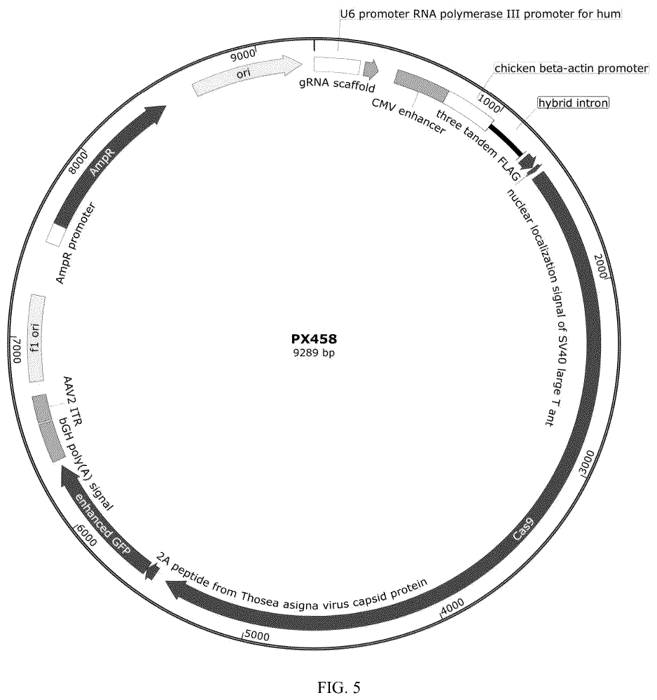

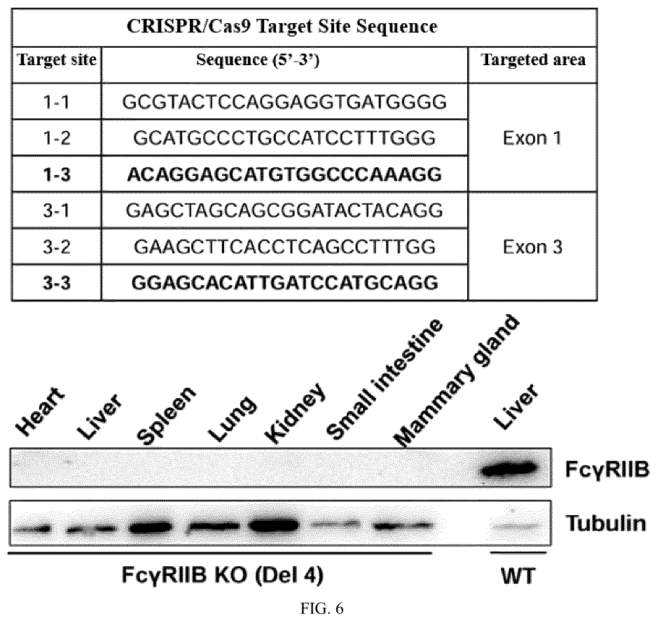

The expression of FcgammaRIIB in mammary gland of pigs during the perinatal period was examined. The results showed that FcgammaRIIB was highly expressed in mammary gland of pigs before parturition and decreased after parturition (see FIG. 4). FcgammaRIIB knockout pigs were subsequently constructed using CRISPR/Cas9 and somatic cell nuclear transfer techniques. The construction method includes cloning sgRNAs into PX458 vector (vector map shown in FIG. 5) and electroporating into healthy, rapidly growing porcine ear fibroblasts. Genomic DNA was extracted from the cells, and primers were designed near the FCGR2B target sites. High-fidelity PCR was performed to amplify genomic sequences containing the target sites, followed by TA cloning. Approximately 20 single colonies per target site were picked for PCR detection. Colonies with correct band size were sent for sequencing. The mutation types were recorded, and the proportion of each mutation type relative to the total number of sequenced clones was calculated to identify highly efficient sgRNAs, namely 1-3 and 3-3 (see FIG. 6:1-3: 5′-ACAGGAGCATGTGGCCCAAAGG-3′ (SEQ UID NO. 15); 3-3: 5′-GGAGCACATTGATCCATGCAGG-3′ (SEQ UID NO. 16)). Single-clone cell lines were then established using these two sgRNA target sites. The recombinant PX458 plasmids containing the two gRNAs were electroporated into fetal ear fibroblasts of male and female pigs of Wuzhishan breed. After 48 h of culture, cells were collected to form a cell suspension and sorted by flow cytometry using the GFP signal carried by PX458. The sorted cells were cultured by limiting dilution at 150-200 cells per 10 cm dish. After approximately 10 days, when the cells formed single clones, clones with regular edges and vigorous growth were picked using a cell cloning ring and transferred to 96-well plates. When the cells reached 80% confluence, the cells were sequentially passaged into 48-well, 24-well, 12-well, and 6-well plates. The portions of the cells were used for genomic DNA extraction to identify mutation types, and remaining cells were cryopreserved. After screening to obtain single-clone cell lines targeting exon 1 and exon 3 of the FCGR2B gene from female and male pigs, cell lines with biallelic knockout and identical mutation types on both chromosomes were selected for somatic cell nuclear transfer. The donor cells for nuclear transfer were all of Wuzhishan breed, and cloned pigs were subsequently obtained. Western blotting confirmed successful deletion of FcgammaRIIB in various tissues of the cloned pigs (see FIG. 6).

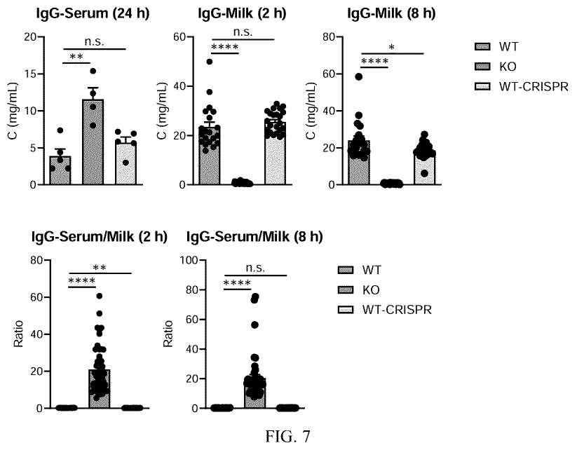

IgG content in postpartum serum and milk of FcgammaRIIB knockout sows was measured by sandwich ELISA. The ratio of serum IgG to milk IgG was analyzed to evaluate milk IgG transport. The results showed that serum IgG content in the knockout pigs was significantly increased, whereas milk IgG was nearly undetectable. The ratio of serum IgG to milk IgG was markedly increased, indicating that FcgammaRIIB serves as the receptor mediating transport of pig serum IgG into milk (see FIG. 7).

### Example 4: Preparation of FcgammaRIIB Overexpressing Mice and Detection of Milk IgG Transport

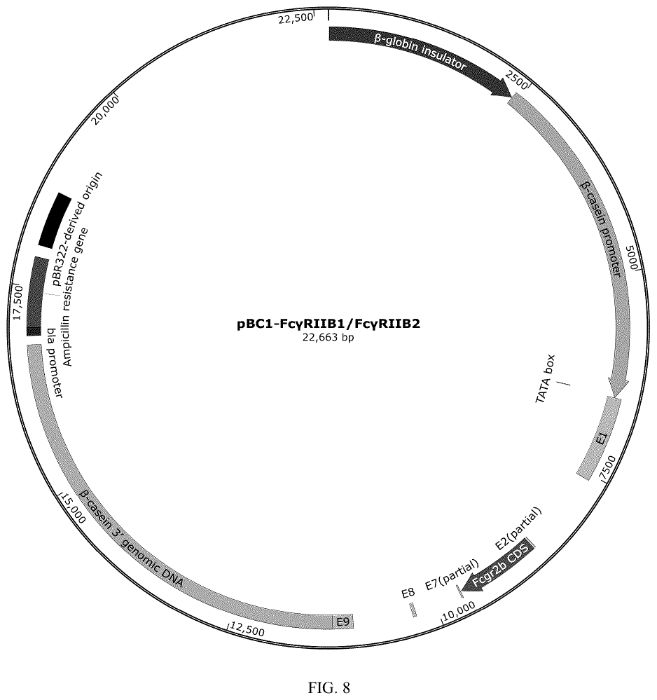

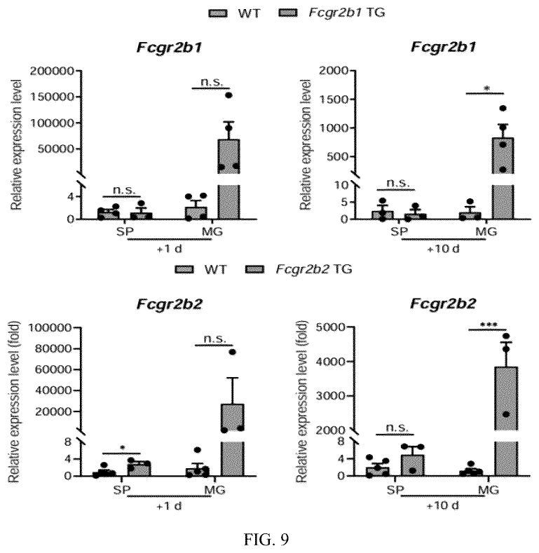

The pBC1 expression vector enables specific overexpression of exogenous genes in mammary gland of mice. Therefore, pBC1-FcgammaRIIB1 and pBC1-FcgammaRIIB2 expression vectors were constructed using restriction enzyme ligation (see FIG. 8). The expression vectors were linearized by Sal I/Not I double digestion and microinjected, resulting in successful generation of F0 transgenic mice. qPCR analysis confirmed successful overexpression of FcgammaRIIB in mammary gland of the transgenic mice (see FIG. 9).

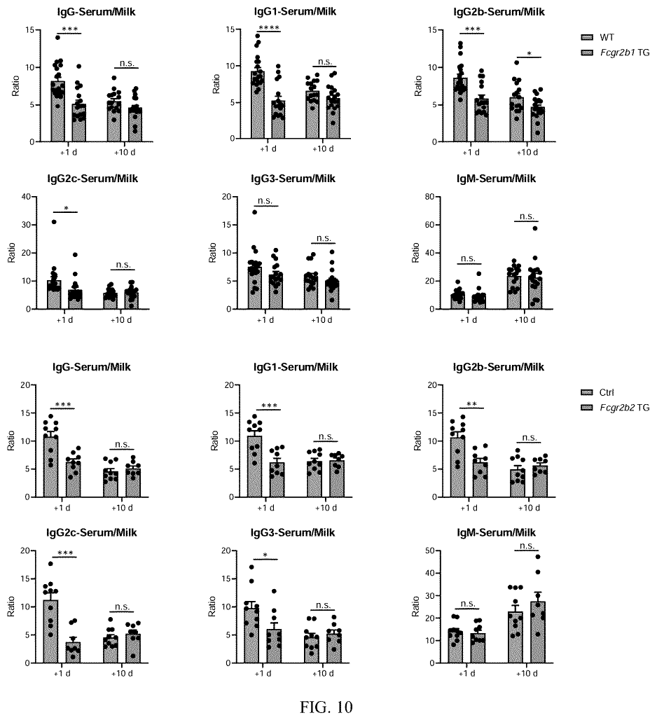

IgG content in postpartum serum and milk of FcgammaRIIB overexpressing mice was measured by sandwich ELISA. The ratio of serum IgG to milk IgG was analyzed to evaluate milk IgG transport. The results showed that serum IgG content in the FcgammaRIIB overexpressing mice remained nearly unchanged, whereas milk IgG content was significantly increased. The ratio of serum IgG to milk IgG was significantly decreased, indicating enrichment of IgG in milk (see FIG. 10).

In summary, the present application demonstrates for the first time that FcgammaRIIB serves as the receptor mediating transport of mammalian serum IgG across the mammary epithelial barrier into milk, which addresses a long-standing unresolved issue in the field of maternal passive immunity. Furthermore, the present application provides a new strategy for increasing colostrum IgG content, thereby enhancing early immunity and survival rate of newborn livestock.

The above mentioned contents are only preferred embodiments of the present application and are not intended to limit the present application. Any modification, equivalent substitution, improvement and the like made within the spirit and principle of the present application shall all fall within the scope of protection of the present application.

What is claimed is:

1. Use of an FcgammaRIIB gene or a protein encoded thereby as an IgG transport receptor in mammary gland of mammals, wherein the FcgammaRIIB gene or the protein encoded thereby mediates transport of mammalian serum IgG across the mammary gland into milk.

2. The use according to claim 1, wherein the mammals comprise mouse, rat, pig, cow, and sheep.

3. The use according to claim 2, wherein mouse FcgammaRIIB has a nucleotide sequence set forth in SEQ ID NO. 1; rat FcgammaRIIB has a nucleotide sequence set forth in SEQ ID NO. 2; pig FcgammaRIIB has a nucleotide sequence set forth in SEQ ID NO. 3; cow FcgammaRIIB has a nucleotide sequence set forth in SEQ ID NO. 4; sheep FcgammaRIIB has a nucleotide sequence set forth in SEQ ID NO. 5;
a protein encoded by the mouse FcgammaRIIB has an amino acid sequence set forth in SEQ ID NO. 6; a protein encoded by the rat FcgammaRIIB has an amino acid sequence set forth in SEQ ID NO. 7; a protein encoded by the pig FcgammaRIIB has an amino acid sequence set forth in SEQ ID NO. 8; a protein encoded by the cow FcgammaRIIB has an amino acid sequence set forth in SEQ ID NO. 9; and a protein encoded by the sheep FcgammaRIIB has an amino acid sequence set forth in SEQ ID NO. 10.

4. The use according to claim 1, wherein the IgG comprises at least one of mouse IgG subtypes IgG1, IgG2a, IgG2b, IgG2c, and IgG3; the IgG comprises at least one of rat IgG subtypes IgG1, IgG2a, IgG2b, and IgG2c; the IgG comprises at least one of pig IgG subtypes IgG1, IgG2, IgG3, IgG4, and IgG5; the IgG comprises at least one of cow IgG subtypes IgG1 and IgG2; and the IgG comprises at least one of sheep IgG subtypes IgG1, IgG2, and IgG3.

5. A method for increasing IgG content in mammalian milk, comprising overexpressing FcgammaRIIB in mammary tissue to increase IgG content in mammalian milk.

6. The method according to claim 5, wherein the overexpression of FcgammaRIIB comprises the following steps: constructing an FcgammaRIIB mammary-specific overexpression vector, followed by microinjection of the overexpression vector.

7. The method according to claim 6, wherein a backbone of the overexpression vector is a pBC1 expression vector.


SEQUENCE LISTING (16 sequences)

SEQ ID NO: 1 - DNA, 1023 bp, synthetic construct
  source 1..1023: mol_type = other DNA
atgggaatcctgccgttcctactgatccccatggagagcaactggactgtccatgtgttctcacggactttgtgccatatgctactgtggacagccgtgctaaatcttgctgctgggactcatgatcttccaaaggctgtggtcaaactcgagcccccgtggatccaggtgctcaaggaagacacggtgacactgacatgcgaagggacccacaaccctgggaactcttctacccagtggttccacaatgggaggtccatccggagccaggtccaagccagctacacgtttaaggccacagtcaatgacagtggagaatatcggtgtcaaatggagcagacccgcctcagcgaccctgtagatctgggagtgatttctgactggctgctgctccagacccctcagctggtgtttctggaaggggaaaccatcacgctaaggtgccatagctggaggaacaaactactgaacaggatctcgttcttccataatgaaaaatccgtgaggtatcatcactacagtagtaatttctctatcccaaaagccaaccacagtcacagtggggactactactgcaaaggaagtctaggaaggacactgcaccagtccaagcctgtcaccatcactgtccaagggcccaagtccagcaggtctttaccagtattgacaattgtggctgctgtcactgggattgctgtcgcagccattgttattatcctagtatccttggtctatctcaagaaaaagcaggttccagctctcccaggaaaccctgatcacagggaaatgggagaaacccttccagaggaagtaggtgagtacagacagccctctgggggctcagtgcctgtcagcccagggcctccatctggactggagccaacaagcagcagcccatacaatcctcctgatctggaagaagctgccaaaactgaggctgagaatacgatcacctactcacttctcaagcatcccgaagccctggatgaagaaacagagcatgattaccagaaccacatttag

SEQ ID NO: 2 - DNA, 888 bp, synthetic construct
  source 1..888: mol_type = other DNA
atgggaaccctgctgttcctaccgctccccatggacagcaacaggactgtcgtccatgtgctctcacggactttgtaccatatgctactgtggacagctgtgctaaatcttgttgctgagtctcatgcaggtcttccgaaggctgtggtgaaacttgagcccccatggatccaggtgcttaaggaagacactgtgacactgatgtgcgaagggacccacaacaccaagaactgttctactcagtggtttcacaatgggagctccatctggcatcaagcccaagccaactacacgtttaaggccacagtcaatgatagtggagaataccggtgccgaatggaggagaccggcatcagcgaacccatacatctgggagtgatttctgactggctgctactccagacctctcaactggtgtttgaagaaggggaaaccatcacattaaggtgccatagctggaagaacaaacagctgactaaggtcttgttgttccagaatggaaaacctgtgaggtattatcatcaaagcagcaatttttcaatccccaaagccaaccacagtcacagtgggaactactattgcaaagcatatctaggaaggacaatgcacgtgtccaagcctgtcaccatcactgtccaagagcccaaatccagcagttcgttgccggtattgacgattgtagctgctgtcgctggaattgctgtagctgccattgttattatcctggtatccttggtctatctcaagaaaaagcaggttccagacactccttctggtctcgaagaagctgagaaaaatgaggtggagaatacaatcacctattcacttctcaagcatcctgaagctccggatgaagaatcagatcacgactaccagaaccacatttag

SEQ ID NO: 3 - DNA, 894 bp, synthetic construct
  source 1..894: mol_type = other DNA
atggggatcccctcgttcctagccctccccgctaccaggagtgactgggctgattgcatgccctgccatcctttgggccacatgctcctgtggacagctctgctattcctggctcctgttcctgggacacatgcagttctcccaaaggctgaggtgaagcttcagcctgcatggatcaatgtgctccaggaggattctgtgacactgacgtgccagggcgaccatgaccctgggaacaccaccacccagtggttccataatgggaacttcacctggaccgagaaccagcccagcttcagctttaaggccaggagagctagcagcggacactacaggtgccagactgcctactccagtctcagcgaccctgtgcatctggatgtgatttctgactggctgctgctccaaacctctagtctggttttccaggaaggggagcccatcttgctgaggtgccatagctggagaaacaagcctctgcataaggttgcatttttccagaatggaaaatctaagaagttttcctatgtggattccaacttctccatcccacaagcaaaccagagtcatagtggcgagtaccactgcacaggatttatcgggaagatgtcgtactcatcacagtctgtgaacatcactgtccaagggtccagcttgaacaacttgctggtgacgatagttgtggctgtggttgcttggattgttgcaatggctattgctgctgccatagcagcctggttccgcctcaggcgaaagcggatttcagccaatcacactgatgctgaagaggctgccaaaattgaggctgaggatacaatcacctattcacttctcttgcatccggaagctgcagaaggagaagcagaggcttcagattaccagaaccacatttaa

SEQ ID NO: 4 - DNA, 891 bp, synthetic construct
  source 1..891: mol_type = other DNA
atggggatcccctcattcctagccttccctgctgccaggaggaaccgagctcactgcacgccctggcatccttggggccacatgctactgtggacagctctgctcttcctggctcctgtttctgggaaacctgatctcccaaaagctgtggtgaccatccagcctgcgtggatcaatgtgctcagggaggatcacgtgacgctgacgtgccaggggaccagcttctctgcaggcaacctcaccacatggttccataacgggagctccatccacacccagaagcagcccagctacagctttagggccggcagcaacgacagtgggtcctacaggtgccagagggagcagaccagcctcagcgaccctgtgcatctggatgtgatttccgactggctgttgctccagacccccagcctcgtgttccaggaaggggagcccatcatgctgaggtgccacagctggagaaaccagcctctgaataagatcacattctaccaggataggaaatccaagatattttcctatcagcgcaccaacttctctatcccacgcgccaacctcagtcacagcggccagtaccactgcacagcgtttatcgggaagatgctacactcgtcacaaccagtgaacatcactgtccaagagtccagctcgagcggcccctcatcgatgacagctgtggctataggcacctgttttgctgcagtggctattgttgcggccataataacctggttccgcctcaggagaaagccaatctcagccggtctcactgatgctgaaaatgatgctgccagaactgaggctgagaacacagtcacctattcactcctctcgcacccggatgttgcagaggaagactcagagtccgattaccagaaacgcctttag

SEQ ID NO: 5 - DNA, 894 bp, synthetic construct
  source 1..894: mol_type = other DNA
atggggatctcctcattcctagccttccctgctgcctggaggaaccgagctcactgcatgccctggcatccttggggccacatgctactgtggacagctctgctcttcccggctcctgttcctgggaaacctgatctcccaaaagctgtggtgaacatccagcctgcgtggatcaatgtgctcagggaggatcacgtgacgctgatgtgccaggggaccagcttctatgcaggcaacctcaccacgtggttccataacgggagctccatccatacccagaaccagcccagctacagctttagggccaacagcaatgacagtggatcctacaggtgccagagggagcagaccagcctcagcgaccctgtgcatctggatgtgatttctgactggctgctgctccagacccccagcctcgtgttccaagaaggggagcccatcatgctgaggtgccacagctggagaaaccaccctctgagtaagatcacgttctaccaggatgggaaatccaagacattttcctatctgcgctccaacttctctatcccacgcgccaacctcagtcacagcggccagtaccgctgcacagcgtttcttgggaagacgccacactcgtcacagcccgtgaacatcactgtccaagagtccagctcgagtgacccctcgtcgacgacgacagctgtggctataggcgcttgttttgctgcagtggctgttgttgctgccatagcagcctggttccgcctcaggagaaagccaatttcagccggtctcactgatgctgaaactgatgctgccagaactgaggctgagaacacaatcacctattcactcctctcgcacccggatgttgcagaggaagacccagagtccgattaccagaaatgcctttag

SEQ ID NO: 6 - protein, 340 aa, synthetic construct
MGILPFLLIPMESNWTVHVFSRTLCHMLLWTAVLNLAAGTHDLPKAVVKLEPPWIQVLKEDTVTLTCEGTHNPGNSSTQWFHNGRSIRSQVQASYTFKATVNDSGEYRCQMEQTRLSDPVDLGVISDWLLLQTPQLVFLEGETITLRCHSWRNKLLNRISFFHNEKSVRYHHYSSNFSIPKANHSHSGDYYCKGSLGRTLHQSKPVTITVQGPKSSRSLPVLTIVAAVTGIAVAAIVIILVSLVYLKKKQVPALPGNPDHREMGETLPEEVGEYRQPSGGSVPVSPGPPSGLEPTSSSPYNPPDLEEAAKTEAENTITYSLLKHPEALDEETEHDYQNHI

SEQ ID NO: 7 - protein, 295 aa, synthetic construct
MGTLLFLPLPMDSNRTVVHVLSRTLYHMLLWTAVLNLVAESHAGLPKAVVKLEPPWIQVLKEDTVTLMCEGTHNTKNCSTQWFHNGSSIWHQAQANYTFKATVNDSGEYRCRMEETGISEPIHLGVISDWLLLQTSQLVFEEGETITLRCHSWKNKQLTKVLLFQNGKPVRYYHQSSNFSIPKANHSHSGNYYCKAYLGRTMHVSKPVTITVQEPKSSSSLPVLTIVAAVAGIAVAAIVIILVSLVYLKKKQVPDTPSGLEEAEKNEVENTITYSLLKHPEAPDEESDHDYQNHI

SEQ ID NO: 8 - protein, 297 aa, synthetic construct
MGIPSFLALPATRSDWADCMPCHPLGHMLLWTALLFLAPVPGTHAVLPKAEVKLQPAWINVLQEDSVTLTCQGDHDPGNTTTQWFHNGNFTWTENQPSFSFKARRASSGHYRCQTAYSSLSDPVHLDVISDWLLLQTSSLVFQEGEPILLRCHSWRNKPLHKVAFFQNGKSKKFSYVDSNFSIPQANQSHSGEYHCTGFIGKMSYSSQSVNITVQGSSLNNLLVTIVVAVVAWIVAMAIAAAIAAWFRLRRKRISANHTDAEEAAKIEAEDTITYSLLLHPEAAEGEAEASDYQNHI

SEQ ID NO: 9 - protein, 296 aa, synthetic construct
MGIPSFLAFPAARRNRAHCTPWHPWGHMLLWTALLFLAPVSGKPDLPKAVVTIQPAWINVLREDHVTLTCQGTSFSAGNLTTWFHNGSSIHTQKQPSYSFRAGSNDSGSYRCQREQTSLSDPVHLDVISDWLLLQTPSLVFQEGEPIMLRCHSWRNQPLNKITFYQDRKSKIFSYQRTNFSIPRANLSHSGQYHCTAFIGKMLHSSQPVNITVQESSSSGPSSMTAVAIGTCFAAVAIVAAIITWFRLRRKPISAGLTDAENDAARTEAENTVTYSLLSHPDVAEEDSESDYQKRL

SEQ ID NO: 10 - protein, 297 aa, synthetic construct
MGISSFLAFPAAWRNRAHCMPWHPWGHMLLWTALLFPAPVPGKPDLPKAVVNIQPAWINVLREDHVTLMCQGTSFYAGNLTTWFHNGSSIHTQNQPSYSFRANSNDSGSYRCQREQTSLSDPVHLDVISDWLLLQTPSLVFQEGEPIMLRCHSWRNHPLSKITFYQDGKSKTFSYLRSNFSIPRANLSHSGQYRCTAFLGKTPHSSQPVNITVQESSSSDPSSTTTAVAIGACFAAVAVVAAIAAWFRLRRKPISAGLTDAETDAARTEAENTITYSLLSHPDVAEEDPESDYQKCL

SEQ ID NO: 11 - DNA, 20 bp, synthetic construct
  source 1..20: mol_type = other DNA
gatacttgcatcgtatcctt

SEQ ID NO: 12 - DNA, 20 bp, synthetic construct
  source 1..20: mol_type = other DNA
ctgacacgcaggccgtccct

SEQ ID NO: 13 - DNA, 20 bp, synthetic construct
  source 1..20: mol_type = other DNA
atggaacatgactctgtcga

SEQ ID NO: 14 - DNA, 20 bp, synthetic construct
  source 1..20: mol_type = other DNA
gagccggttgataatatctg

SEQ ID NO: 15 - DNA, 22 bp, synthetic construct
  source 1..22: mol_type = other DNA
acaggagcatgtggcccaaagg

SEQ ID NO: 16 - DNA, 22 bp, synthetic construct
  source 1..22: mol_type = other DNA
ggagcacattgatccatgcagg